(19) United States
(12) Patent Application Publication
(10) Pub. No.: US 2012/0172397 A1
(43) Pub. Date: Jul. 5, 2012

(54) HEXAHYDROCYCLOPENTA[f]INDAZOLE 5-YL ETHANOLS AND DERIVATIVES THEREOF AS SELECTIVE GLUCOCORTICOID RECEPTOR MODULATORS

(75) Inventors: Helen J. Mitchell, Richboro, PA (US); Danielle M. Hurzy, Havertown, PA (US); Robert S. Meissner, Devon, PA (US)

(73) Assignee: MERCK SHARP & DOHME CORP., Rahway, NJ (US)

(21) Appl. No.: 13/392,014

(22) PCT Filed: Aug. 30, 2010
(86) PCT No.: PCT/US10/47119

§ 371 Date: Feb. 23, 2012

Related U.S. Application Data
(60) Provisional application No. 61/240,381, filed on Sep. 8, 2009.

(51) Int. Cl. A61K 31/444; A61K 31/4439; C07D 401/12; A61P 35/02; A61P 25/00; A61P 3/10; A61P 5/00; A61P 3/04; A61P 3/00; A61P 1/00; A61P 19/02; A61P 11/00; A61P 1/04; A61P 1/16; A61P 17/06; A61P 17/00; A61P 31/22; A61P 31/18; A61P 35/00; A61P 25/24; A61P 25/30; A61P 25/18; A61P 25/22; C07D 213/02

(52) U.S. Cl. 514/333; 546/275.7; 514/338; 546/256

(57) ABSTRACT

The present invention encompasses compounds of Formula (I) or pharmaceutically acceptable salts or hydrates thereof, which are useful as selective glucocorticoid receptor ligands for treating a variety of autoimmune and inflammatory diseases or conditions. Pharmaceutical compositions and methods of use are also included.

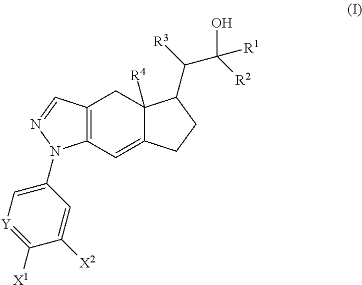

### BACKGROUND OF THE INVENTION

Intracellular receptors (IR's) are a class of structurally related proteins involved in the regulation of gene expression. The steroid hormone receptors are a subset of this superfamily whose natural ligands are typically comprised of endogenous steroids such as estradiol, progesterone, and cortisol. Man-made ligands to these receptors play an important role in human health and, of these receptors, the glucocorticoid receptor has an essential role in regulating human physiology and immune response. Steroids that interact with the glucocorticoid receptor have been shown to be potent anti-inflammatory agents. The present invention is directed to a novel class of compounds that are selective glucocorticoid receptor modulators that have potent anti-inflammatory and immunosuppressive activity and possess advantages over steroidal glucocorticoid ligands with respect to side effects, efficacy, toxicity and/or metabolism.

### SUMMARY OF THE INVENTION

The present invention encompasses compounds of Formula I:

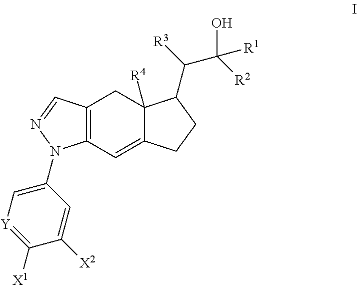

or pharmaceutically acceptable salts or hydrates thereof, which are useful as selective glucocorticoid receptor ligands for treating a variety of autoimmune and inflammatory diseases or conditions. Pharmaceutical compositions and methods of use are also included.

### DETAILED DESCRIPTION OF THE INVENTION

The present invention encompasses a compound of Formula I,

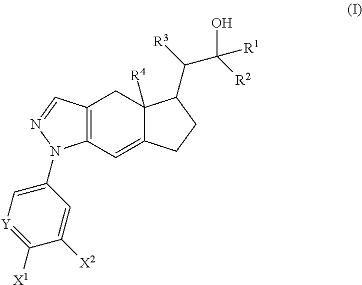

or a pharmaceutically acceptable salt thereof, or a stereoisomer thereof, or a pharmaceutically acceptable salt of the stereoisomer thereof:
wherein
each of X¹ and X² is independently hydrogen or halogen;
Y is carbon or nitrogen, provided that when Y is nitrogen, then both X¹ and X² are hydrogen;
each of R¹ and R² is independently selected from the group consisting of:
(1) hydrogen,
(2) C₁₋₆alkyl,
(3) C₃₋₆cycloalkyl,
(4) C₁₋₆alkyl-aryl,
(5) C₁₋₆alkyl-HET,
(6) aryl, and
(7) HET,
wherein each of items (2) to (3), items (6) to (7), the aryl portion of item (4), and
   the HET portion of item (5) above is optionally substituted with one to three substituents independently selected from the group consisting of:
   (a) halogen,
   (b) C₁₋₆alkyl,
   (c) C₁₋₆alkyl-halogen,
   (d) —OR^{a},
   (e) oxo,
   (f) —C(O)NR^{a}R^{b},
   (g) —NR^{a}R^{b}, and
   (h) —SO₂R^{a};
      R³ is hydrogen or C₁₋₆alkyl;
      R⁴ is C₁₋₆alkyl or C₃₋₆cycloalkyl;
      each of R^{a} and R^{b} is independently selected from the group consisting of:
(1) hydrogen,
(2) C₁₋₆alkyl,
(3) aryl, and
(4) HET; and
   each occurrence of HET is independently selected from the group consisting of:
(1) a 5- or 6-membered aromatic or non-aromatic monocyclic ring containing 1-3 heteroatoms selected from O, S and N, and
(2) a 9- or 10-membered aromatic or partially aromatic bicyclic ring containing 1-3 heteroatoms selected from O, S, and N.

In one embodiment of compounds of Formula I, each of X¹ and X² is independently hydrogen or halogen.

In another embodiment of compounds of Formula I, both X¹ and X² are hydrogen.

In one embodiment of compounds of Formula I, Y is carbon or nitrogen.

In another embodiment, Y is nitrogen, and both X¹ and X² are hydrogen.

In one embodiment, compounds disclosed herein have Formula Ia:

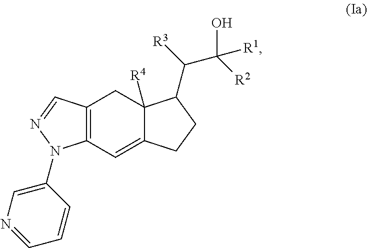

wherein R¹, R², R³ and. R⁴ are as defined above under Formula I.

In one embodiment of compounds of Formula I or Ia, each of R¹ and R² is independently selected from the group consisting of:
(1) hydrogen,
(2) C₁₋₄alkyl,
(3) C₃₋₆cycloalkyl,
(4) C₁₋₄alkyl-phenyl,
(5) C₁₋₄alkyl-HET,
(6) phenyl, and
(7) HET,
wherein each of items (2), (3), (6) and (7), the phenyl portion of item (4), and the HET portion of item (5) above is optionally substituted with one to three substituents independently selected from the group consisting of:
   (a) halogen,
   (b) C₁₋₄alkyl,
   (c) C₁₋₄alkyl-halogen,
   (d) —OR¹, and
   (e) oxo.

In another embodiment, R¹ is hydrogen or C₁₋₄alkyl, and

R2 is selected from the group consisting of:
(1) C₁₋₄alkyl,
(2) C₃₋₆cycloalkyl,
(3) C₁₋₄alkyl-phenyl,
(4) C₁₋₄alkyl-HET,
(5) phenyl, and
(6) HET,
wherein each of items (1), (2), (5) and (6), the phenyl portion of item (3), and the
   HET portion of item (4) above is optionally substituted with one to three substituents independently selected from the group consisting of:
   (a) halogen,
   (b) C₁₋₄alkyl,
   (c) C₁₋₄alkyl-halogen,
   (d) —OR^{a}, and
   (e) oxo.

In one embodiment of compounds of Formula I or Ia, R³ is hydrogen or C₁₋₄alkyl. In another embodiment, R³ is hydrogen. In yet another embodiment, R3 is methyl or ethyl.

In one embodiment of compounds of Formula I or Ia, R⁴ is C ₁₋₄alkyl. In another embodiment, R⁴ is methyl or ethyl. In yet another embodiment, R⁴ is methyl.

In one embodiment of compounds of Formula I or Ia, each of R^{a} and R^{b} is independently hydrogen or C₁₋₄alkyl. In another embodiment, each of R^{a} and R^{b} is independently hydrogen or methyl. In yet another embodiment, each of R^{a} and R^{b} is hydrogen.

In one embodiment of compounds of Formula I or Ia, aryl is phenyl.

In one embodiment of compounds of Formula I or Ia, each occurrence of HET is independently selected from the group consisting of:
(1) a 5- or 6-membered aromatic or non-aromatic monocyclic ring containing 1-3 heteroatoms selected from O, S and N, and
(2) a 9- or 10-membered aromatic or partially aromatic bicyclic ring containing 1-3 heteroatoms selected from O, S, and N.

In another embodiment, each occurrence of HET is independently a 5- or 6-membered aromatic or non-aromatic monocyclic ring containing 1-3 heteroatoms selected from O, S and N.

In another embodiment, each occurrence of HET is independently a 9- or 10-membered aromatic or partially aromatic bicyclic ring containing 1-3 heteroatoms selected from O, S, and N.

In yet another embodiment, each occurrence of HET is independently selected from the group consisting of: pyridine, pyrimidine, pyridazine, furan, thiophene, thiazole, oxazole, isooxazole, benzofuran, benzothiophene, indole, pyranopyrrole, benzopyran, quionoline, benzocyclohexyl, naphtyridine, benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuxanyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzirnidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetratolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

In another embodiment, each occurrence of HET is independently selected from the group consisting of: pyridine, pyrimidine, pyridazine, pyrrolyl, furan, oxazole, isooxazole, benzofuran, indole, benzopyran, dihydrobenzofuranyl, dihydrobenzoxazolyl, dihydrofuranyl, dihydroindolyl, dihydroisooxazolyl, dihydropyridinyl, dihydropyrimidinyl, and dihydropyrrolyl.

In still another embodiment, each occurrence of HET is independently selected from the group consisting of pyridine, pyrimidine, pyridazine, furan, thiophene, thiazole, oxazole, and isooxazole.

In one embodiment, the compounds disclosed herein have Formula Ib:

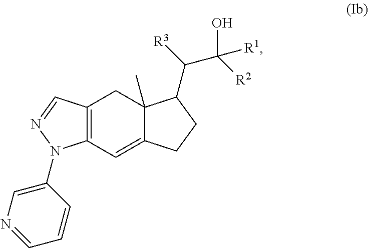

wherein
each of R¹ and R2 is independently selected from the group consisting of:
(1) hydrogen,
(2) C₁₋₄alkyl,
(3) C₃₋₆cycloalkyl,
(4) C₁₋₄alkyl-phenyl,
(5) C₁₋₄alkyl-HET,
(6) phenyl, and
(7) HET,
wherein each of items (2) to (3), items (6) to (7), the phenyl portion of item (4), and the HET portion of item (5) above is optionally substituted with one to three substituents independently selected from the group consisting of:
   (a) halogen,
   (b) C₁₋₄alkyl,
   (c) C₁₋₄alkyl-halogen,
   (d) —OR^{a},
   (e) oxo,
   (f) —C(O)NR^{a}R^{b},
   (g) —NR^{a}R^{b}, and
   (h) —SO₂R^{a};
      R³ is hydrogen or C₁₋₄alkyl;
      each of R^{a} and R^{b} is independently selected from the group consisting of:
(1) hydrogen,
(2) C₁₋₄alkyl,
(3) phenyl, and
(4) HET; and
   each occurrence of HET is independently selected from the group consisting of: pyridine, pyrimidine, pyridazine, pyrrolyl, furan, oxazole, isooxazole, benzofuran, indole, benzopyran, dihydrobenzofuranyl, dihydrobenzoxazolyl, dihydrofuranyl, dihydroindolyl, dihydroisooxazolyl, dihydropyridinyl, dihydropyrimidinyl, and dihydropyrrolyl.

In one embodiment of Formula Ib, R¹ is hydrogen or methyl, and R² is selected from the group consisting of:
(1) C₁₋₄alkyl,
(2) C₁₋₄alkyl-phenyl,
(3) C₁₋₄alkyl-HET,
(4) phenyl, and
(5) HET,
wherein each of items (1), (4) and (5), the phenyl portion of item (2), and the HET portion of item (3) above is optionally substituted with one to three substituents independently selected from the group consisting of:
   (a) halogen,
   (b) C₁₋₄alkyl,
   (c) C₁₋₄alkyl-halogen,
   (d) —OR^{a}, and
   (e) oxo.

In one embodiment of Formula Tb, each occurrence of HET is independently selected from the group consisting of: pyridine, pyrimidine, pyridazine, furan, oxazole, benzofuran, indole, and benzopyran.

In one embodiment of Formula Ib, each of R^{a} and R^{b} is independently hydrogen or methyl.

In one embodiment, the invention encompasses a pharmaceutical composition comprising a compound disclosed herein in combination with a pharmaceutically acceptable carrier.

Another embodiment of the invention encompasses a method for treating a glucocorticoid receptor mediated disease or condition in a mammalian patient in need of such treatment comprising administering to the patient a compound disclosed herein in an amount that is effective for treating the glucocorticoid receptor mediated disease or condition.

Within this embodiment is encompassed the above method wherein the glucocorticoid receptor mediated disease or condition is selected from the group consisting of tissue rejection, leukemias, lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity, metabolic syndrome, inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, cirrhosis, inflammatory scalp alopecia, panniculitis, psoriasis, discoid lupus erythematosus, inflamed cysts, atopic dermatitis, pyoderma gangrenosum, pemphigus vulgaris, buflous pemphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma, Human Immunodeficiency Virus (HIV), cell apoptosis, cancer, Kaposi's sarcoma, retinitis pigmentosa, cognitive performance, memory and learning enhancement, depression, addiction, mood disorders, chronic fatigue syndrome, schizophrenia, sleep disorders, and anxiety.

Another embodiment of the invention encompasses the use of a compound disclosed herein for treating of a glucocorticoid receptor mediated disease or condition in a mammalian patient in need of such treatment.

Another embodiment of the invention encompasses a method of selectively modulating the activation, repression, agonism and antagonism effects of the glucocorticoid receptor in a mammal comprising administering to the mammal a compound disclosed herein in an amount that is effective to modulate the glucocorticoid receptor.

Another embodiment of the invention encompasses the use of a compound disclosed herein for selectively modulating the activation, repression, agonism and antagonism effects of the glucocorticoid receptor in a mammal.

The invention is described using the following definitions unless otherwise indicated.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl, cyclopropyl, cyclobutyl, cyclopentyl and cyclohexyl.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes , propenyl, 1-methylethenyl, butenyl and the like.

The term “cycloalkyl” means mono-, bi- or tri-cyclic structures, optionally combined with linear or branched structures, the indicated number of carbon atoms. Examples of cycloalkyl groups include cyclopropyl, cyclopentyl, cycloheptyl, adamantyl, cyclododecylmethyl, 2-ethyl-1- bicyclo[4.4.0]decyl, and the like.

The term “aryl” is defined as a mono- or bi-cyclic aromatic ring system and includes, for example, phenyl, naphthyl, and the like.

The term “optionally substituted” means “unsubstituted or substituted,” and therefore, the generic structural formulas described herein encompass compounds containing the specified optional substituent as well as compounds that do not contain the optional substituent. Each variable is independently defined each time it occurs within the generic structural formula definitions.

The term “HET” is defined as a 5- to 10-membered aromatic, partially aromatic or non-aromatic mono- or bicyclic ring, containing 1-4 heteroatoms selected from O, S and N, and optionally substituted with 1-2 oxo groups. Preferably, “HET” is a 5- or 6-membered aromatic or non-aromatic monocyclic ring containing 1-3 heteroatoms selected from O, S and N, for example, pyridine, pyrimidine, pyridazine, furan, thiophene, thiazole, oxazole, isooxazole and the like, or HET is a 9- or 10-membered aromatic or partially aromatic bicyclic ring containing 1-3 heteroatoms selected from O, S, and N, for example, benzofuran, benzothiophene, indole, pyranopyrrole, benzopyran, quionoline, benzocyclohexyl, naphtyridine and the like. “HET” also includes the following: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

For all of the above definitions, each reference to a group is independent of all other references to the same group when referred to in the Specification. For example, if both R¹ and R² are HET, the definitions of HET are independent of each other and R¹ and R² may be different HET groups, for example furan and pyridine.

The term “treating” encompasses not only treating a patient to relieve the patient of the signs and symptoms of the disease or condition but also prophylactically treating an asymptomatic patient to prevent the onset of the disease or condition or preventing, slowing or reversing the progression of the disease or condition. The term “amount effective for treating” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician.

### Optical Isomers-Diastereomers-Geometric Isomers-Tautomers

Compounds described herein may contain an asymmetric center and may thus exist as enantiomers. Where the compounds according to the invention possess two or more asymmetric centers, they may additionally exist as diastereomers. When bonds to the chiral carbon are depicted as straight lines in the formulas of the invention, it is understood that both the (R) and (S) configurations of the chiral carbon, and hence both enantiomers and mixtures thereof, are embraced within the formulas. The present invention includes all such possible stereoisomers as substantially pure resolved enantiomers, racemic mixtures thereof, as well as mixtures of diastereomers. The present invention includes all stereoisomers of the compounds disclosed herein and pharmaceutically acceptable salts thereof.

Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound disclosed herein may be obtained by stereospecific synthesis using optically pure starting materials or reagents of known configuration.

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH₂C(O)-groups (keto forms) may undergo tautomerism to form hydroxyl —CH=C(OH)-groups (enol forms). Both keto and enol forms, individually as well as mixtures thereof, are included within the scope of the present invention.

### Isotopes

In the compounds disclosed herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds disclosed herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds disclosed herein can be prepared without undue experimentation by conventional techniques well known to those skilled in the art or by processes analogous to those described in the Schemes and Examples herein using appropriate isotopically-enriched reagents and/or intermediates.

### Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabarnine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, mane, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

### Solvates

The present invention includes within its scope solvates of the compounds disclosed herein. As used herein, the term “solvate” refers to a complex of variable stoichiometry formed by a solute (i.e., a compound of Formula I or Ia) or a pharmaceutically acceptable salt thereof and a solvent that does not interfere with the biological activity of the solute. Examples of solvents include, but are not limited to water, ethanol, and acetic acid. When the solvent is water, the solvate is known as hydrate; hydrates include, but are not limited to, hemi-, mono, sesqui-, di- and trihydrates.

### Prodrugs

The present invention includes within its scope the use prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with a compound of Formula I, Ia, or Ib, or with a compound which may not be a compound of Formula I, Ia, or Ib, but which converts to a compound of Formula I, Ia, or Ib in vivo after administration to the patient. Conventional procedures for the selection and preparation of suitable prodrug derivatives are described, for example, in “Design of Prodrugs,” ed. H. Bundgaard, Elsevier, 1985.

The pharmaceutical compositions of the present invention comprise a compound disclosed herein as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that in the discussion of methods of treatment which follows, references to the compounds disclosed herein are meant to also include the pharmaceutically acceptable salts.

The magnitude of prophylactic or therapeutic dose of a compound disclosed herein, will vary with the nature and the severity of the condition to be treated and with the particular compound and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for oral administration to humans may contain from about 0.5 mg to about 5 g of active agent compounded with an appropriate and convenient amount of carrier material which may vary from about 5 to about 95 percent of the total composition. Dosage unit forms will generally contain from about 1 mg to about 2 g of an active ingredient, typically 25 mg, 50 mg, 100 mg, 200 mg, 300 mg, 400 mg, 500 mg, 600 mg, 800 mg, or 1000 mg.

For the treatment of glucocorticoid receptor mediated diseases the compound disclosed herein may be administered orally, topically, parenterally, by inhalation spray or rectally in dosage unit formulations containing conventional non-toxic pharmaceutically acceptable carriers, adjuvants and vehicles. The term parenteral as used herein includes subcutaneous, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, solutions, aqueous or oily suspensions, dispersible powders or granules, emulsions, hard or soft capsules, syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a perservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds disclosed herein may also be administered in the faun of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing a compound disclosed herein are employed. (For purposes of this application, topical application shall include mouth washes and gargles.) Topical formulations may generally be comprised of a pharmaceutical carrier, cosolvent, emulsifier, penetration enhancer, preservative system, and emollient.

The ability of the compounds disclosed herein to selectively modulate glucocorticoid receptors makes them useful for treating, preventing or reversing the progression of a variety of inflammatory and autoimmune diseases and conditions. Thus, the compounds of the present invention are useful to treat, prevent or ameliorate the following diseases or conditions: inflammation, tissue rejection, auto-immunity, various malianancies, such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity and metabolic syndrome.

The compounds disclosed herein are also useful for treating, preventing or reversing the progression of disease states involving systemic inflammation such as inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, and cirrhosis.

The compounds disclosed herein are useful for treating, preventing or reversing the progression of a variety of topical diseases such as inflammatory scalp alopecia, panniculitis, psoriasis, discoid lupus erythematosus, inflamed cysts, atopic dermatitis, pyoderma gangrenosum, pemphigus vulgaris, buflous pemphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma.

The compounds disclosed herein are also useful in treating, preventing or reversing the progression of disease states associated with Human Immunodeficiency Virus (HIV), cell apoptosis, and cancer including, but not limited to, Kaposi's sarcoma, immune system activation and modulation, desensitization of inflammatory responses, IIL-I expression, natural killer cell development, lymphocytic leukemia, and treatment of retinitis pigmentosa. Cognitive and behavioral processes are also susceptible to glucocorticoid therapy where antagonists would potentially be useful in the treatment of processes such as cognitive performance, memory and learning enhancement, depression, addiction, mood disorders, chronic fatigue syndrome, schizophrenia, stroke, sleep disorders, and anxiety.

The invention also encompasses a method for treating a glucocorticoid receptor mediated disease comprising concomitantly administering to a patient in need of such treatment a compound disclosed herein and one or additional more agents. For treating or preventing asthma or chronic obstructive pulmonary disease, the compounds disclosed herein may be combined with one or more agents selected from the group consisting of: θ-agonists (e.g., salmeterol), theophylline, anticholinergics (e.g., atropine and ipratropium bromide), cromolyn, nedocromil and leukotriene modifiers (e.g., montelukast). For treating or preventing inflammation, the compounds disclosed herein may be combined with one or the following: a salicylate, including acetylsalicylic acid, a non-steroidal antiinflammatory drug, including indomethacin, sulindac, mefenamic, meclofenamic, tolfenamic, tolmetin, ketorolac, dicofenac, ibuprofen, naproxen, fenoprofen, ketoprofen, flurbiprofin and oxaprozin, a TNF inhibitor, including etanercept and infliximab, an IL-1 receptor antagonist, a cytotoxic or immunosuppressive drug, including methotrexate, leflunomide, azathioprine and cyclosporine, a gold compound, hydroxychloroquine or sulfasalazine, penicillamine, darbufelone, and a ρ38 kinase inhibitor. The compound disclosed herein may also be used in combination with bisphonates such as alendronate to treat a glucocorticoid mediated disease and simultaneously inhibit osteoclast-mediated bone resorption.

Unless noted otherwise, the following abbreviations have the indicated meanings:
b.p.=boiling point
e.q.=equivalent(s)
EtOAc=ethyl acetate
g=gram(s)
mg=milligram(s)
min.=minute(s)
mL=milliliter(s)
mol.=mole(s)
mmol.=millimole(s)
m.p.=melting point
mol. wt.=molecular weight
L=liter(s)
r.t.=room temperature
THF=tetrahydrofuran
v=volume
w=weight

### Alkyl Group Abbreviations

Me=methyl
Et=ethyl
n-Pr=normal propyl
i-Pr=isopropyl
n-Bu=normal butyl
i-Bu=isobutyl
s-Bu=secondary butyl
t-Bu=tertiary butyl
c-Pr=cyclopropyl
c-Bu=cyclobutyl
c-Pen=cyclopentyl
c-Hex=cyclohexyl

### METHODS OF SYNTHESIS

Generally, compounds of the present invention may be synthesized by using the following synthetic scheme and examples:

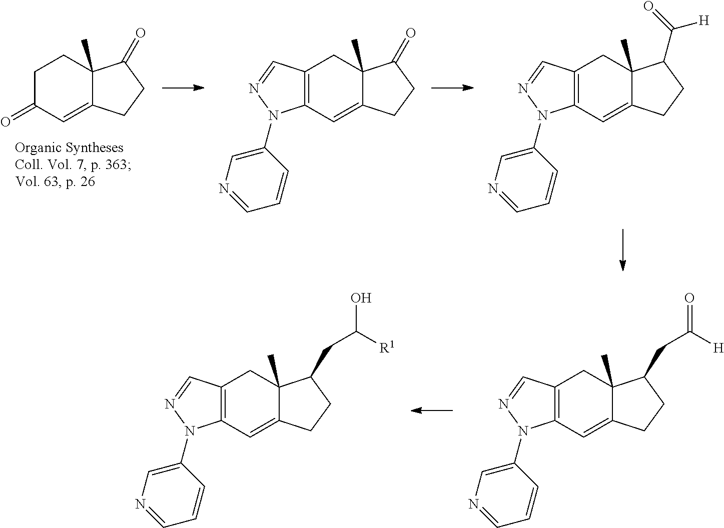

Unless noted otherwise, the following synthetic conditions were used for the preparation of the following examples:
(i) all operations were carried out at room or ambient temperature, that is, at a temperature in the range 18-25° C.,
(ii) evaporation of solvent was carried out using a rotary evaporator under reduced pressure (600-4000 pascals: 4.5-30 mm Hg) with a bath temperature of up to 60° C.,
(iii) the course of reactions was followed by thin layer chromatography (TLC) and reaction times are given for illustration only;
(iv) melting points are uncorrected and ‘d’ indicates decomposition; the melting points given are those obtained for the materials prepared as described; polymorphism may result in isolation of materials with different melting points in some preparations;
(v) the structure and purity of all final products were assured by at least one of the following techniques: TLC, mass spectrometry, nuclear magnetic resonance (NMR) spectrometry or micro-analytical data;
(vi) yields are given for illustration only;
(vii) when given, NMR data is in the form of delta (δ) values for major diagnostic protons, given in parts per million (ppm) relative to tetramethylsilane (TMS) as internal standard, determined at 500 MHz or 600 MHz using the indicated solvent, conventional abbreviations used for signal shape are: s. singlet; d. doublet; t. triplet; m. multiplet; and br. broad. In addition, “Ar” means an aromatic signal.

The reaction schemes and examples described herein illustrate the methods employed in the synthesis of the compounds of the present invention. In some cases the order of carrying out the reaction schemes may be varied to facilitate the reaction or to avoid unwanted reaction products. The synthesis of the novel compounds which are the subject of this invention may be accomplished by one or more of several similar routes. The following examples are provided so that the invention can be more fully understood. These examples are illustrative only and should not be construed as limiting the invention in any way.

### EXAMPLES 1a and 1b

### Diastereoisomers

2-[(4αR,5R)-4α-methyl-1-(pyridin-3-yl)-1,4,4α,5,6,7-hexahydrocyclopenta[ƒ]indazol-5-yl]-1-phenylethanol

As described in more detail below, 1-11a and 1-11b were separated at Step I with the TIP

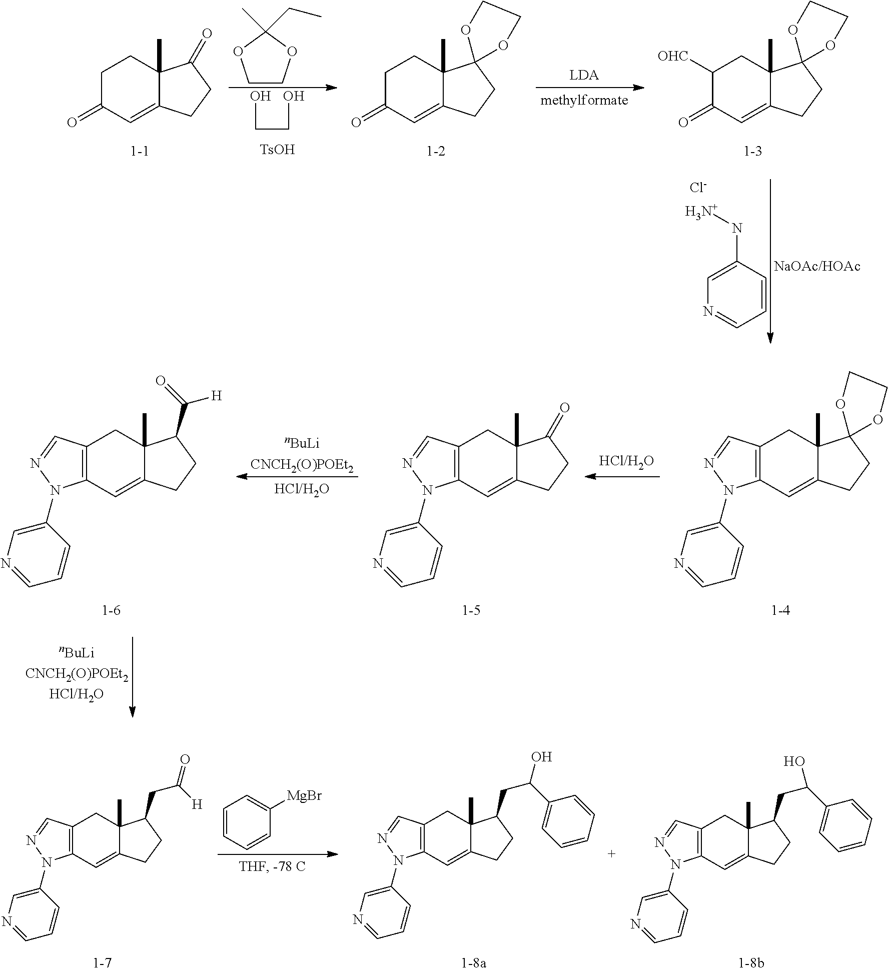

(7α′S)-7α′-Methyl-2′,3′,7′,7α′-tetrahydrospiro[1,3-dioxolane-2,1′-inden]-5′(6′H)-one (1-2′

### Step A:

Ethylene glycol (12.2 mL, 219 mmol) and p-toluenesulfonic acid monohydrate (4.40 g, 25.6 mmol) were added to a solution of Hajos-Parrish Ketone (See Organic Syntheses, Coll. Vol. 7, p.363; Vol 63, p.26) (1-1, 60.0 g, 365 mmol) in 2-ethyl-2-methyl-1,3-dioxolan (46 mL) and the resulting solution stirred at ambient temperature for 24 hours. The reaction was poured into saturated aqueous NaHCO₃ solution (1 L) and the crude product extracted with EtOAc (×3). The combined organic extracts were dried over anhydrous MgSO4 and the solvent removed in vacuo. Purification by flash chromatography on 1.5 kg of silica, eluting with a gradient of 0-70% EtOAc in hexanes afforded 48.5 g, 64% of 1-2 as a clear viscous oil. MS (ESI): m/z =209.3 (MH+).

### Step B:

(7α′S-7α′-Methyl-5′-oxo-2′,3′,5′,6′,7′,7α′-hexahydrospiro[1,3-dioxolane-2,1′-indene]-6′-carbaldehyde (1-3):

A 1.5 M solution of lithium diisopropylamide mono(tetrahydrofuran) in cyclohexane (465 mL, 0.698 mol) was added to a solution of 1-2 (48.5 g, 0.233 mol) in diethyl ether (930 mL) at -78 ° C. and the resulting solution stirred at this temperature for 1 hour to afford a thick suspension. Methyl formate (86.6 mL, 1.40 mol) was added dropwise over about 30 min and the resulting suspension stirred at -78° C. for 5 hours. The reaction was quenched at -78° C. with 1 M aqueous HCl solution (3 L) and the aqueous layer checked to ensure it was acidic. The crude product was extracted with EtOAc (×3) and the combined organic extracts were dried over anhydrous MgSO₄ and the solvent removed in vacuo to afford 60 g of crude 1-3 (74% pure) as a tan viscous oil that was used directly in the next step without purification. MS (ESI): m/z=237.3 (MH+).

### Step C:

(4αS)-1-(3-pyridyl)-4α-methyl-4,4α,6,7-tetrahydrocyclopenta[ƒ]indazol-5(1H)-one (1-5):

Sodium acetate (2.0 g, 24.4 mmol) was added to a solution of crude 1-3 (5.23 g, 22.1 mmol), 3-pyridylhydrazine bis-hydrochloride (4.0 g, 22.1 mmol) and acetic acid (89 mL) in toluene (100 mL) and the resulting suspension was stirred at room temperature for 1 hour. The reaction was diluted with EtOAc, and washed carefully (CO₂ evolution) with aqueous 5% w/v NaHCO₃ solution (2×1 L), then dried over anhydrous MgSO₄ and concentrated to afford a viscous crude brown oil. The crude oil was dissolved in THF (100 mL) and aqueous 4 M HCl (30 mL) was added and the resulting solution was heated to 65° C. for 30 min. The resulting solution was cooled to ambient temperature, and poured slowly into aqueous 5% w/v NaHCO₃ solution (CO₂ evolution), and the crude product was extracted with EtOAc (×3). The combined organic extracts were dried over anhydrous MgSO₄ and the solvent removed in vacua. Purification by flash chromatography on 300 g of silica, eluting with a gradient of 0-100% EtOAc in hexanes afforded 3.93 g of 1-5, as a brown solid. MS (ESI): m/z=266.2 (MH+).

### Step D:

(4αR,5S)-4α-methyl-1-(pyridin-3-yl)-1,4,4α,5,6,7-hexahydrocyclopenta[ƒ]indazole-5-carbaldehyde (1-6):

A 1.6 M solution of ⁿBuLi in hexanes (12.75 mL, 31.9 mmol) was added to a solution of diethyl isocyanomethylphosphonate (5.11 mL, 31.9 mmol) in anhydrous THF (15 mL) at −78° C. and the resulting solution stirred at this temperature for 30 min. A solution of 1-5 (4.7 g, 17.7 mmol) in anhydrous THF (10 mL) was added dropwise over about 20 min and the resulting solution was stirred at −78° C. for 1 hour, then warmed directly to ambient temperature and stirred for a further 1 hour. The reaction was quenched with saturated aqueous ammonium chloride solution, and the crude product was extracted with EtOAc (3×). The combined organic extracts were dried over anhydrous MgSO₄ and the solvent removed in vacua. The vinyl isocyanate intermediate was dissolved in diethyl ether (50 mL) and 4 M aqueous HCl (30 mL) was then added and the biphasic mixture was stirred at ambient temperature for 12 hours. The reaction was quenched with 1 M aqueous HCl, and the crude product was extracted with EtOAc (3×). The combined organic extracts were dried over anhydrous MgSO₄ and the solvent removed in vacuo. Purification by flash chromatography on 300 g of silica, eluting with a gradient of 0-100% EtOAc in hexanes afforded 0.6 g of the product 1-6 as a yellow oil.

MS (ESI): m/z=280.2 (MH+).

### Step E:

[(4αR,5R)-4α-methyl-1-(pyridine-3-yl)-1,4,4α,5,6,7-hexahydrocyclopenta[ƒ]indazole-5-yl]acetaldehyde (1-7):

A 1.6 M solution of ⁿBuLi in hexanes (1.55 mL, 3.87 mmol) was added to a solution of diethyl isocyanomethylphosphonate (0.62 mL, 3.87 mmol) in anhydrous THF (5 mL) at −78° C. and the resulting solution stirred at this temperature for 30 min. A solution of 1-6 (0.60 g, 2.15 mmol) in anhydrous THF (3 mL) was added dropwise over about 20 min and the resulting solution was stirred at −78° C. for 1 hour, then warmed directly to ambient temperature and stirred for a further 1 hour. The reaction was quenched with saturated aqueous ammonium chloride solution, and the crude product was extracted with EtOAc (3×). The combined organic extracts were dried over anhydrous MgSO₄ and the solvent removed in vacuo. The vinyl isocyanate intermediate was dissolved in diethyl ether (10 mL) and 4 M aqueous HCl (10 mL) was then added and the biphasic mixture was stirred at ambient temperature for 12 hours. The reaction was quenched with 1 M aqueous HCl, and the crude product was extracted with EtOAc (3×). The combined organic extracts were dried over anhydrous MgSO₄ and the solvent removed in vacuo. Purification by flash chromatography on 120 g of silica, eluting with a gradient of 0-100% EtOAc in hexanes afforded 0.6 g of the product 1-7 as a yellow oil.

MS (ESI): m/z=294.2 (MH+).

### Step F:

2-[(4αR,5R)-4α-methyl-1-(pyridine-3-yl)-1,4,4a,5,6,7-hexahydrocyclopenta[ƒ]indazol-5-yl]-1-phenylethanol (1-8a and 1-8b):

Phenylmagnesium bromide (0.68 mL, 0.68 mmol, 1.0 M solution in THF) was added to a cooled, stirred solution of 1-7 (0.10 g, 0.34 mmol) in anhydrous THF (2 mL) at −78° C. The mixture was stirred at −78° C. for 30 min and then warmed to room temperature and stirred for 30 min. The reaction was quenched by the addition of saturated aqueous ammonium chloride, diluted with EtOAc and washed with H₂O, and brine. The combined organic extracts were dried over anhydrous MgSO₄ and the solvent removed in vacuo. Purification by GILSON HPLC on reverse phase silica gel, eluting with a gradient of 5-60% acetonitrile in water afforded the two diastereoisomers 1-8a and 1-8b as white foams.

1-8a: HRMS (APCI): m/z=372.2071 (MH+).
1-8b: HRMS (APCI): m/z=372.2073 (MH+).

The following Examples 2a-6b in Table 1 were prepared following the general synthetic schemes and procedures analogous to Examples 1a and 1b described above. In some cases (e.g. Examples 2a and 2b), the two diastereoisomers were separated at the last step after the TIPS group was removed using standard chromatographic techniques (Hexanes-Ethyl Acetate on silica gel, or Acetonitrile-water on reverse phase HPLC).

TABLE 1

| Ex. # | STRUCTURE | NAME | M + 1 |
|---|---|---|---|
| 1a | 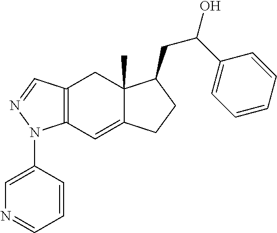 | 2-[(4αR,5R)-4α-methyl-1-(pyridin-3-yl)- 1,4,4α,5,6,7-hexahydrocyclopenta[f] indazol-5-yl]-1-phenylethanol | 372.2071 |
| 1b | 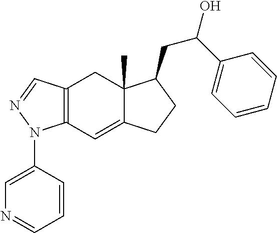 | 2-[(4αR,5R)-4α-methyl-1-(pyridin-3-yl)- 1,4,4α,5,6,7-hexahydrocyclopenta[f] indazol-5-yl]-1-phenylethanol | 372.2073 |
| 2a | 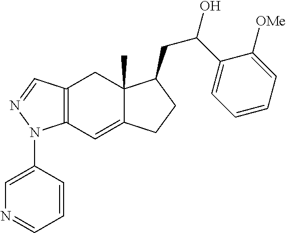 | 1-(2-methoxyphenyl)-2-[(4αR,5R)-4α- methyl-1-(pyridin-3-yl)-1,4,4α,5,6,7- hexahydrocyclopenta[f]indazol-5-yl] ethanol | 402.2171 |
| 2b | 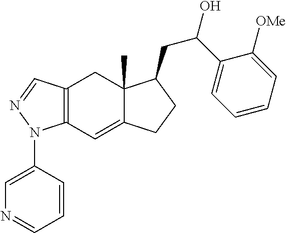 | 1-(2-methoxyphenyl)-2-[(4αR,5R)-4α- methyl-1-(pyridin-3-yl)-1,4,4α,5,6,7- hexahydrocyclopenta[f]indazol-5-yl] ethanol | 402.2172 |
| 3 | 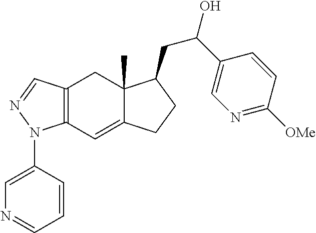 | 1-(6-methoxypyridin-3-yl)-2-[(4αR,5R)- 4α-methyl-1-(pyridin-3-yl)-1,4,4α,5,6,7- hexahydrocyclopenta[f]indazol-5-yl] ethanol | 403.2121 |
| 4a | 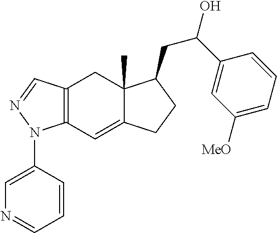 | 1-(3-methoxyphenyl)-2-[(4αR,5R)-4α- methyl-1-(pyridin-3-yl)-1,4,4α,5,6,7- hexahydrocyclopenta[f]indazol-5-yl] ethanol | 402.2182 |
| 4b | 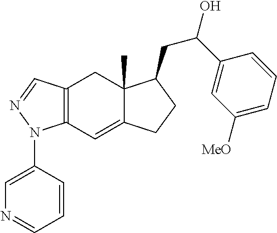 | 1-(3-methoxyphenyl)-2-[(4αR,5R)-4α- methyl-1-(pyridin-3-yl)-1,4,4α,5,6,7- hexahydrocyclopenta[f]indazol-5-yl] ethanol | 402.2182 |
| 5 | 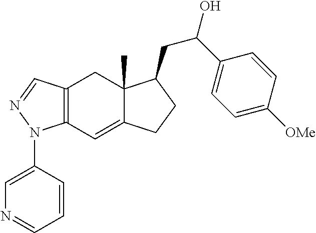 | 1-(4-methoxyphenyl)-2-[(4αR,5R)-4α- methyl-1-(pyridin-3-yl)-1,4,4α,5,6,7- hexahydrocyclopenta[f]indazol-5-yl] ethanol | 402.2182 |
| 6a | 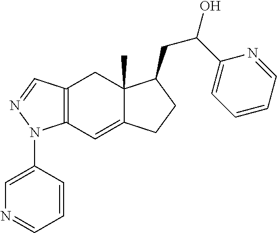 | 2-[(4αR,5R)-4α-methyl-1-(pyridin-3- yl)-1,4,4α,5,6,7-hexahydrocyclopenta[f] indazol-5-yl]-1-(pyridin-2-yl)ethanol | 373.2026 |
| 6b | 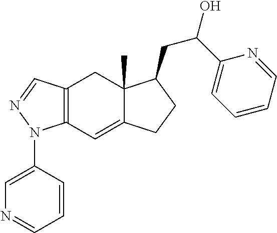 | 2-[(4αR,5R)-4α-methyl-1-(pyridin-3- yl)-1,4,4α,5,6,7-hexahydrocyclopenta[f] indazol-5-yl]-1-(pyridin-2-yl)ethanol | 373.2026 |

### EXAMPLE 7

1-[(4αR,5R)-4α-methyl-1-(pyridin-3-yl)-1,4,4α,5,6,7-hexahydrocyclopenta[ƒ]indazol-5-yl]-2-phenylpropan-2-ol (7a and 7b):

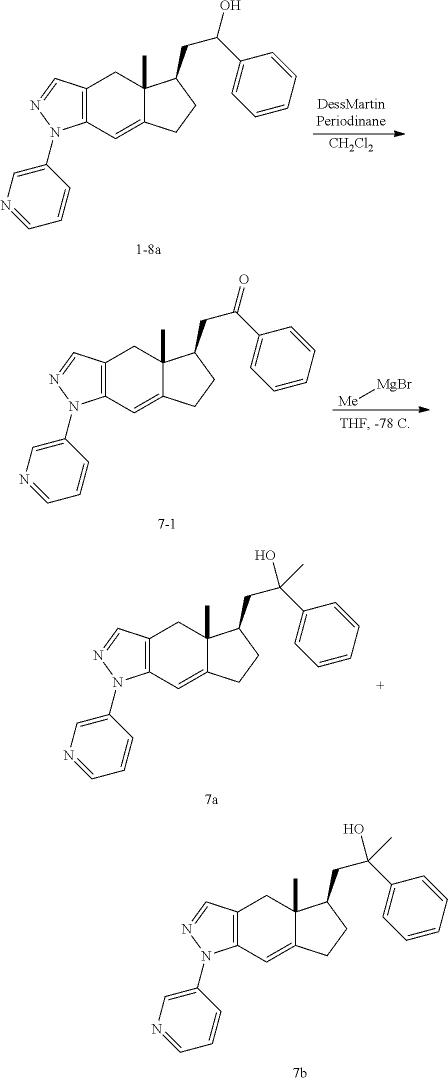

### Step A:

[(4αR,5R)-4αmethyl-1-(pyridin-3-yl)-1,4,4α,5,6,7-hexahydrocyclopenta[ƒ]indazol-5-yl]-1-phenylethanone (7-1)

Dess-Martin Periodinane (1.009 g, 2.38 mmol) was added to a solution of 1-8a/1-8b (0.68 g, 1.83 mmol) in anhydrous CH₂Cl₂ (2 mL) at room temperature and the resulting solution stirred for 30 min. The reaction was quenched with saturated aqueous sodium thiosulfate and diluted CH₂Cl₂. The resulting mixture was stirred for 10 min and then filtered through a pad of celite. The organic layer was washed with saturated aqueous sodium bicarbonate, dried over anhydrous MgSO4 and the solvent removed in vacuo to afford the crude product 7-1 (0.86 g) as a yellow oil. MS (ESI): m/z=370.2 (MH+).

### Step B:

1-[(4αR,5R)-4α-methyl-1-(pyridin-3-yl)-1,4.4α,5,6,7-hexahydrocyclopenta[ƒ]indazol-5-yl]-2-phenylpropan-2-ol (7a and 7b)

Methylmagnesium bromide (0.136 mL, 0.41 mmol, 1.0 M solution in THF) was added to a cooled, stirred solution of 2-1 (0.05 g, 0.135 mmol) in anhydrous THF (1 mL) at −78° C. The mixture was stirred at −78° C. for 30 min and then warmed to room temperature and stirred for 30 mins. The reaction was quenched by the addition of saturated aqueous ammonium chloride, diluted with EtOAc and washed with H₂O, and brine. The combined organic extracts were dried over anhydrous MgSO₄ and the solvent removed in vacua. Purification by GILSON HPLC on reverse phase silica gel, eluting with a gradient of 10-60% acetonitrile in water afforded the two diastereoisomers 7a and 7b as white foams.

7a: HRMS (APCI): m/z=386.2283 (MH+).
7b: HRMS (APCI): m/z=386.2283 (MH+).

### BIOLOGICAL ASSAYS

The compounds exemplified in the present application exhibited activity in one or more of the following assays.

### GR Ligand Binding Assay

### Materials:

Binding Buffer: TEGM (10 mM Tris-HCl, 1 mM EDTA, 10% glycerol, 1 rnM beta-mecaptoethanol, 10 mM Sodium Molybdate, pH 7.2)

50% HAP Slurry: Calbiochem Hydroxylapatite, Fast Flow, in 10 mM Iris, pH 8.0 and 1 mM EDTA.

Wash Buffer: 40 mM Tris, pH7.5, 100 mM KCl, 1 mM EDTA and 1 mM EGTA. 95% EtOH

Dexmethasone-methyl-³H, (DEX*); (Amersham cat# TRK645)

Dexamethasone(DEX) (Sigma, cat# D1756):

Hydroxylapatite Fast Flow; Calbiochem Cat#391947

Molybdate=Molybdic Acid (Sigma, M1651)

### HeLa Cell Culture Media:

| RPMI 1640 (Gibco 11835-055) w/23.8 | Final conc. | |
|---|---|---|
| mM NaHCO₃, 2 mM L-glutamine | | |
| in 500 mL of complete media | | |
| 10 mL (1M Hepes) | 20 | mM |
| 5 mL (200 mM L-glu) | 4 | mM |
| 0.5 mL (10 mg/mL human insulin) | 10 | μg/mL |
| in 0.01N HCl | | |
| Calbiochem#407694-S) | | |
| 50 mL FBS (Sigma F2442) | 10% | |
| 1 mL (10 mg/mL Gentamicin | 20 | μg/mL |
| Gibco#15710-072) | | |

### Cell Passaging

Cells (Hall R. E., et al., European Journal of Cancer, 30A: 484-490 (1994)) HeLa (ATCC) cultured in RPMI 1640 (Gibco 11835-055) containing 20 mM Hepes, 4 mM L-glu, 10 μg/ml of human insulin (Sigma, I-0259), 10% FBS and 20 μg/ml of Gentamicin (Gibco#15710-072) are rinsed twice in PBS. Phenol red-free Trypsin-EDTA is diluted in the same PBS 1:10. The cell layers are rinsed with 1× Trypsin, extra Trypsin is poured out, and the cell layers are incubated at 37° C. for ˜2 min. The flask is tapped and checked for signs of cell detachment. Once the cells begin to slide off the flask, the complete media is added. The cells are counted at this point, then diluted to the appropriate concentration and split into flasks or dishes for further culturing (Usually 1:3 to 1:6 dilution).

### Preparation of HeLa Cell Lysate

When the cells are 70 to 85% confluent, they are detached as described above, and collected by centrifuging at 1000 g for 10 minutes at 4° C. The cell pellet is washed twice with TEGM (10 mM Tris-HCl, 1 mM EDTA, 10% glycerol, 1 mM beta-mercaptoethanol, 10 mM Sodium Molybdate, pH 7.2). After the final wash, the cells are resuspended in TEGM at a concentration of 10⁷ cells/mL. The cell suspension is snap frozen in liquid nitrogen or ethanol/dry ice bath and transferred to −80° C. freezer on dry ice. Before setting up the binding assay, the frozen samples are left on ice-water to just thaw (˜1 hr). Then the samples are centrifuged at 12,500 g to 20,000 g for 30 min at 4° C. The supernatant is used to set-up assay right away. If using 50 μL of supernatant, the test compound can be prepared in 50 μL of the TEGM buffer.

### Procedure for Multiple Compound Screening

1× TEGM buffer is prepared, and the isotope-containing assay mixture is prepared in the following order: EtOH (2% final concentration in reaction), ³H-DEX (Amersham Biosciences) and 1× TEGM. [e.g. For 100 samples, 200 μL (100×2) of EtOH+4.25 μL of 1:10 ³H-Dex stock+2300 μL (100×23) 1× TEGM]. The compound is serially diluted, e.g., if starting final conc. is 1 μM, and the compound is in 25 μL of solution, for duplicate samples, 75 μL of 4×1 μM solution is made and 3 μL of 100 μM is added to 72 μL of buffer, and 1:5 serial dilution.

25 μL of ³H-DEX (6 nM) trace and 25 μL compound solution are first mixed together, followed by addition of 50 μL receptor solution. The reaction is gently mixed, spun briefly at about 200 rpm and incubated at 4° C. overnight. 100 μL of 50% HAP slurry is prepared and added to the incubated reaction which is then vortexed and incubated on ice for 5 to 10 minutes. The reaction mixture is vortexed twice more to resuspend HAP while incubating reaction. The samples in 96-well format are then washed in wash buffer using The FilterMate™ Universal Harvester plate washer (Packard). The washing process transfers HAP pellet containing ligand-bound expressed receptor to Unifilter-96 GF/B filter plate (Packard). The HAP pellet on the filter plate is incubated with 50 μL of MICROSCINT (Packard) scintillint for 30 minutes before being counted on the TopCount microscintillation counter (Packard). IC₅₀s are calculated using DEX as a reference.

### Trans-Activation Modulation of Glucocorticoid Receptor Assay

This assay assesses the ability of test compounds to control transcription from the MMTV-LUC reporter gene in lung adenocarcinoma A549 cells or HeLa cells, a human breast cancer cell line that naturally expresses the human GR. The assay measures induction of a modified MMTV LTR/promoter linked to the LUC reporter gene.

The routine transient assay consists of plating 7,000-25,000 cells/well of a white, clear-bottom 96-well plate. Alternatively, 384-well plates can be used at a cell concentration of 10,000 /well. The media that the cells are plated in is “exponential growth medium” which consists of phenol red-free RPMI1640 containing 10%FBS, 4 mM L-glutamine, 20 mM HEPES, 10 μg/mL human insulin, and 20 μg/mL gentamicin. Incubator conditions are 37° C. and 5% CO2. The transfection is done in batch mode. The cells are trypsinized and counted to the right cell number in the proper amount of fresh media. It is then gently mixed with the FuGene6/DNA mix and plated onto the 96 or 384-well plate, all the wells receive 100 μL or 40 μL, respectively, of medium +lipid/DNA complex then incubated 37° C. overnight. The transfection cocktail consists of serum-free OptiMEM, FuGene6 reagent and DNA. The manufacturer's (Roche Biochemical) protocol for cocktail setup is as follows: The lipid to DNA ratio is approximately 2.5:1 and the incubation time is 20 min at room temperature. Sixteen to 24 hours after transfection, the cells are treated with dexamethasone to a final concentration of 10 nM as well as the compound of interest, such that final DMSO (vehicle) concentration is equal to or less than 1%. Each plate also contains samples that are treated with 10 nM dexamethasone alone, which is used as the 100% activity control. The cells are exposed to the compounds for 24 hours. After 24 hours, the cells are lysed by a Promega cell culture lysis buffer for approximately 30 min and then the luciferase activity in the extracts is assayed in the 96-well format luminometer. In 384-well format, Steady-Glo (Promega) or Steady-Lite (PerkinElmer) can be used by adding an equal volume of reagent to the media present in each well. Activity induced by 10 nM dexamethasone alone is set at 100% activity. Antagonist activity is calculated by determining the decrease in dexamethasone-induced activity in response to compound treatment relative to samples that were treated with dexamethasone alone. Results are expressed as % inhibition of 10 nM dexamethasone activity or as fold of 10 nM dexamethasone activity. This transactivation assay can be performed in an agonist and antagonist mode to identify these different activities.

Activity of test compounds is calculated as the Eₘₐₓ relative to the activity obtained with 300 nM dexamethasone. Activity of test compounds is calculated as the Eₘₐₓ relative to the activity obtained with 300 nM DEX. The exemplified tissue selective glucocorticoid receptor modulators of the present invention display agonist activity in this assay of greater than 5% and less than 100%, and maximal transactivation activity less then maximal transrepression activity.

The action of compounds is also tested in an antagonist mode (Anti-GRAMMER) in which the cells are treated with medium containing an agonist such as 10 nM DEX and the ability to agents to inhibit the activation by an agonist is measured.

### Transrepression Assay

This assay assesses the ability of test compounds to control transcription from the TNFα-β-lactamase reporter gene in U937 cells, a human myelomonocytic leukemia cell line that naturally expresses the human GR. The assay measures compound dependent-repression of the TNFa promoter linked to a reporter gene.

The human U937cells that had been stablely transfected with the TNF-α promoter driving β-lactamase are used for this assay. U937 cells contain an endogenous glucocorticoid receptor (GR). Cells are maintained in RPMI 1640 Growth medium (Gibco Cat#11875-093) containing 25 mM HEPES, 10% FBS, 2 mM L-Glutamine, 1 mM Sodium pyruvate, 25 μg/ml Gentamicin (Gibco Cat#15710-064), 1:1000 2-Mercaptoethanol (Gibco Cat#21985-023) and 0.8 mg/ml G418 (Gibco Cat#10131-027). The density of the cells in the flask needs to be about 1×106−3×106/ml at the time of harvest. Usually, the cells are split to 1.2˜1.4×105/ml (1:10) 3 days prior to the assay. 50,000 cells/well are plated in 96 well black-walled plates the day of assay. Test compounds are added 10 μL/well, and cells are incubated at 37° C. for 30-45 min. For assaying compounds, first dilute 1:10 in DMSO to make 1 mM, then further dilute 1:100 in medium to make 10 × stock prior to adding to the cells. Add 50 ng/ml PMA (Sigma, cat# P8139) 10 μL/well to a final concentration 5 ng/ml, and 1 μg/ml LPS (Sigma, cat# L4130) 10 μL/well to a final concentration 100 ng/ml. Incubate cells at 370° C. overnight for˜18 hr. PMA is stored frozen as 100 μg/ml stock in DMSO. Dilute 1:10 in DMSO for a working stock of 10 μg/ml and store at −20° C. For assaying, dilute the 10 μg/ml working stock 1:200 in medium to make a 10× solution (50 ng/ml). Store frozen LPS at 1 mg/ml in PBS, dilute 1:1000 in medium to make 10× (1 μg/ml) for the assay. Add 6× loading buffer (CCF2-AM) 20 μL/well, and incubate at room temperature for 70˜90 min. Read plates on CytoFluor II Plate Reader according to manufacture suggested protocols. The activity repressed by 100 nM dexamethasone alone is set as 100% activity.

Microarray Analysis

This assay assesses the ability of test compounds to modulate the transcription of endogenously expressed genes in a variety of cell types including but not limited to A549, HeLa or U937 cells. All cell culture reagents were purchased from Invitrogen Life Tech, Carlsbad Calif. A549 cells were grown in phenol red-free DMEM/F12 medium supplemented with 10% FBS. Cells were grown at 37° C. with 5% CO2. Using the RNeasy Kit (Qiagen Corp, Valencia Calif.), total RNA was extracted and purified from A549 cells treated with different GC compounds for 24 hours, at a fully active dose. These cells express large amount of the GR and are very responsive to GC treatment. All samples were compared against cells treated with vehicle. Expression levels of 23000 genes were measured using oligonucleotide microarrays purchased from Agilent Technologies, Inc. Each comparison was done on a pair of microarrays with reversed fluorophores. Raw image intensity data were processed according to the method described in U.S. Pat. No. 6,351,712. The method was used to remove dye bias and to derive a Rosetta probability (p) and fold change value for each gene and each sample pair. Furthermore, for each gene an ANOVA model was constructed across all treatments to derive error estimates. P values for evaluating expression differences were computed using a Bayesian adjusted t-test that was developed by Lönnstedt and Speed (2002) and extended by Smyth (2003). A gene was declared differentially expressed in any particular comparison if it satisfied two criteria:

1. The Rosetta p value had to be less than 0.1 and the Rosetta fold change value had to be greater than 1.4 in at least one of the treatments.

2. The ANOVA p value had to be less than 0.01 and the fold change greater than 2 in the comparison under consideration.

### In Vivo Inflammation Assay

Intact adult (6 month old) female Sprague-Dawley rats are used in the oxazolone (OX) contactdermatitis model. Rats were sensitized on the ventral abdomen with OX on Day 0. On Days 7 and 9, a randomly-selected ear was challenged (same ear each time) with OX; the other was treated with vehicle. Daily treatment begun on Day 7 and continued for 7 d with test compounds at different doses and 1.3 mpk 6-methlyprednisolone or 0.1 mpk DEX as positive controls. The thickness of both ears are measured on Days 11 and 14. Necropsy occurred on Day 14. The rat is first weighed, then anesthetized in a CO₂ chamber until near death. Approximately 5 ml whole blood is obtained by cardiac puncture. The rat is then examined for certain signs of death and completeness. Tissues are dissected in a highly stylized fashion. The the following endpoints were evaluated: a) inhibiting ear inflammation induced by oxazolone, b) raising serum insulin, c) reducing serum ACTH, d) reducing spleen weight, e) reducing skin thickness, f) reducing body weight, g) increasing expression of bone-related genes with potential relationship to negative glucocorticoid effects on bone; e) changes in molecular markers that correlate with skin inflammation, skin thinning, muscle atrophy and glucose metabolism in liver. All blood samples were collected between 1330-1530 hours, ˜4-5 hrs after the last compound treatment.

Primary data for this assay are left and right ear thickness. Inter-ear thickness difference (etd) is used for the estimating the level of inflammation and effectiveness of the compounds is determined by their ability to reduce the increase the thickness of the inflamed ear. Back of the rat skin thickness, spleen weight, serum insulin as well as the effects of gcs on the expression of molecular markers in skin inflammation, skin atrophy, muscle atrophy and glucose metabolism in liver are measured. Data are analyzed by anova plus fisher plsd post-hoc test to identify intergroup differences.

### Bioassay Results

The compounds described herein were tested in the Binding, GRAMMER and GITAR assays and demonstrated the activity profiles as shown in Table 2. Compounds shown in Table 2 have potencies in the GRAMMER and GITAR assays (as measured by inflection points, IP) of less than 1000 nM.

TABLE 2

| | | Transactivation | | Transrepression | |
|---|---|---|---|---|---|
| | GR | A549 Cells | | U937 Cells | |
| Example | BIND | GRAMMER | | GITAR | |
| # | Ki (nM) | IP (nM) | Emax (%) | IP (nM) | Emax (%) |
| 1a | 3.5 | 343.4 | 29.7 | 277.0 | 75.4 |
| 1b | 8.8 | 811.3 | 8.5 | 1715.0 | 49.3 |
| 2a | 7.3 | 301.0 | 68.5 | 228.9 | 79.5 |
| 2b | 9.0 | 812.1 | 21.5 | 1157.0 | 73.9 |
| 3 | 8.5 | 159.9 | 101.1 | 200.4 | 85.2 |
| 4a | 3.6 | 472.0 | 47.7 | 114.0 | 74.8 |
| 4b | 8.4 | 797.4 | 15.3 | 547.5 | 47.0 |
| 5 | 0.9 | 53.4 | 90.7 | 20.7 | 87.3 |
| 6a | 345.8 | 1172.0 | 1.6 | 558.1 | 16.6 |
| 6b | 150.5 | 1429.0 | 2.2 | 1383.0 | 30.0 |
| 7a | 2.3 | 74.3 | 53.1 | 78.7 | 84.2 |
| 7b | 4.8 | 315.8 | 20.0 | 393.4 | 65.9 |

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of Formula Ia, or a pharmaceutically acceptable salt thereof, or a stereoisomer thereof, or a pharmaceutically acceptable salt of the stereoisomer thereof: 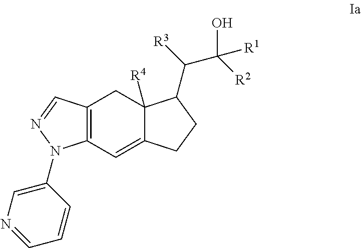
wherein
each of R¹ and R² is independently selected from the group consisting of:
(1) hydrogen,
(2) C₁₋₆alkyl,
(3) C₃₋₆cycloalkyl,
(4) C₁₋₆alkyl-aryl,
(5) C₁₋₆alkyl-HET,
(6) aryl, and
(7) HET,
wherein each of items (2) to (3), items (6) to (7), the aryl portion of item (4), and the HET portion of item (5) above is optionally substituted with one to three substituents independently selected from the group consisting of:
(a) halogen,
(b) C₁₋₆alkyl,
(c) C₁₋₆alkyl-halogen,
(d) —OR^{a},
(e) oxo,
(f) —C(O)NR^{a}R^{b},
(g) —NR^{a}R^{b}, and
(h) —SO₂R^{a};
R³ is hydrogen or C₁₋₆alkyl;
R⁴ is C₁₋₆alkyl or C₃₋₆cycloalkyl;
each of R^{a} and R^{b} is independently selected from the group consisting of:
(1) hydrogen,
(2) C₁₋₆alkyl,
(3) aryl, and
(4) HET; and
each occurrence of HET is independently selected from the group consisting of:
(1) a 5- or 6-membered aromatic or non-aromatic monocyclic ring containing 1-3 heteroatoms selected from O, S and N, and
(2) a 9- or 10-membered aromatic or partially aromatic bicyclic ring containing 1-3 heteroatoms selected from O, S, and N.

2. The compound of claim 1, wherein
R¹ is hydrogen or C₁₋₄alkyl, and
R2 is selected from the group consisting of:
(1) C₁₋₄alkyl,
(2) C₃₋₆cycloalkyl,
(3) C₁₋₄alkyl-phenyl,
(4) C₁₋₄alkyl-HET,
(5) phenyl, and
(6) HET,
wherein each of items (1), (2), (5) and (6), the phenyl portion of item (3), and the HET portion of item (4) above is optionally substituted with one to three substituents independently selected from the group consisting of:
(a) halogen,
(b) C₁₋₄alkyl,
(c) C₁₋₄alkyl-halogen,
(d) —OR^{a}, and
(e) oxo.

3. The compound of claim 1, wherein R4 is methyl or ethyl.

4. The compound of claim 1, wherein each of R^{a} and R^{b} is independently hydrogen or methyl.

5. The compound of claim 1, wherein HET is independently selected from the group consisting of: pyridine, pyrimidine, pyridazine, pyrrolyl, furan, oxazole, isooxazole, benzofuran, indole, benzopyran, dihydrobenzofuranyl, dihydrobenzoxazolyl, dihydrofuranyl, dihydroindolyl, dihydroisooxazolyl, dihydropyridinyl, dihydropyrimidinyl, and dihydropyrrolyl.

6. A compound of Formula Ib, or a pharmaceutically acceptable salt thereof, or a stereoisomer thereof, or a pharmaceutically acceptable salt of the stereoisomer thereof: 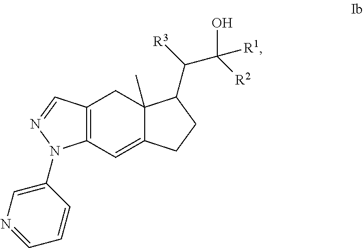
wherein
each of R¹ and R2 is independently selected from the group consisting of:
(1) hydrogen,
(2) C₁₋₄alkyl,
(3) C₃₋₆cycloalkyl,
(4) C₁₋₄alkyl-phenyl,
(5) C₁₋₄alkyl-HET,
(6) phenyl, and
(7) HET,
wherein each of items (2) to (3), items (6) to (7), the phenyl portion of item (4), and the HET portion of item (5) above is optionally substituted with one to three substituents independently selected from the group consisting of:
(a) halogen,
(b) C₁₋₄alkyl,
(c) C₁₋₄alkyl-halogen,
(d) —OR^{a},
(e) oxo,
(f) —C(O)NR^{a}R^{b},
(g) —NR^{a}R^{b}, and
(h) —SO₂R^{a};
R³ is hydrogen or C₁₋₄alkyl;
each of R^{a} and R^{b} is independently selected from the group consisting of:
(1) hydrogen,
(2) C₁₋₄alkyl,
(3) phenyl, and
(4) HET; and
each occurrence of HET is independently selected from the group consisting of: pyridine, pyrimidine, pyridazine, pyrrolyl, furan, oxazole, isooxazole, benzofuran, indole, benzopyran, dihydrobenzofuranyl, dihydrobenzoxazolyl, dihydrofuranyl, dihydroindolyl, dihydroisooxazolyl, dihydropyridinyl, dihydropyrimidinyl, and dihydropyrrolyl.

7. The compound of claim 6, wherein
R¹ is hydrogen or methyl, and
R² is selected from the group consisting of
(1) C₁₋₄alkyl,
(2) C₃₋₆cycloalkyl,
(3) C₁₋₄alkyl-phenyl,
(4) C₁₋₄alkyl-HET,
(5) phenyl, and
(6) HET,
wherein each of items (1), (2), (5) and (6), the phenyl portion of item (3), and the
HET portion of item (4) above is optionally substituted with one to three substituents independently selected from the group consisting of
(a) halogen,
(b) C₁₋₄alkyl,
(c) C₁₋₄alkyl-halogen,
(d) —OR^{a}, and
(e) oxo.

8. The compound of claim 6, wherein each occurrence of HET is independently selected from the group consisting of: pyridine, pyrimidine, pyridazine, furan, oxazole, benzofuran, indole, and benzopyran.

9. The compound of claim 6, wherein each of R^{a} and R^{b} is independently hydrogen or methyl.

10. A compound selected from the group consisting of:
2-[(4αR,5R)-4α-methyl-1-(pyridine-3-yl)-1,4,4a,5,6,7-hexahydrocyclopenta[ƒ]indazol-5-yl]-1-phenylethanol
1-(2-methoxyphenyl)-2-[(4αR,5R)-4α-methyl-1-(pyridin-3-yl)-1,4,4α,5,6,7-hexahydrocyclopenta[ƒ]indazol-5-yl]ethanol,
1-(3-methoxyphenyl)-2-[(4αR,5R)-4α-methyl-1-(pyridin-3-yl)-1,4,4α,5,6,7-hexahydrocyclopenta[ƒ]indazol-5-yl]ethanol,
1-(4-methoxyphenyl)-2-[(4αR,5R)-4α-methyl-1-(pyridin-3-yl)-1,4,4α,5,6,7-hexahydroeyclopenta[ƒ]indazol-5-yl]ethanol,
2-[(4αR,5R)-4α-methyl-1-(pyridine-3-yl)-1,4,4a,5,6,7-hexahydrocyclopenta[ƒ]indazol-5-yl]-1-(pyridin-2-yl)ethanol, and
1-[(4αR,5R)-4α-methyl-1-(pyridin-3-yl)-1,4,4α,5,6,7-hexahydrocyclopenta[ƒ]indazol-5-yl]-2-phenylpropan-2-ol;
or a pharmaceutically acceptable salt thereof, or a stereoisomer thereof, or a pharmaceutically acceptable salt of the stereoisomer thereof.

11. A pharmaceutical composition comprising a compound of claim 1 in combination with a pharmaceutically acceptable carrier.

12. A method for treating a glucocorticoid receptor mediated disease or condition in a mammalian patient in need of such treatment comprising administering to the patient a compound of claim 1 in an amount that is effective for treating the glucocorticoid receptor mediated disease or condition.

13. The method of claim 12 wherein the glucocorticoid receptor mediated disease or condition is selected from the group consisting of tissue rejection, leukemias, lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity, metabolic syndrome, inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, cirrhosis, inflammatory scalp alopecia, panniculitis, psoriasis, discoid lupus erythematosus, inflamed cysts, atopic dermatitis, pyoderma gangrenosum, pemphigus vulgaris, buflous pemphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma, Human Immunodeficiency Virus (HIV), cell apoptosis, cancer, Kaposi's sarcoma, retinitis pigmentosa, cognitive performance, memory and learning enhancement, depression, addiction, mood disorders, chronic fatigue syndrome, schizophrenia, sleep disorders, and anxiety.

14. A method of selectively modulating the activation, repression, agonism or antagonism effect of the glucocorticoid receptor in a mammal comprising administering to the mammal a compound of claim 1 in an amount that is effective to modulate the glucocorticoid receptor.
